(19) United States
(12) Patent Application Publication
(10) Pub. No.: US 2024/0156535 A1
(43) Pub. Date: May 16, 2024

(54) CUSTOMIZED CUT AND SCREW GUIDE AND THE METHOD FOR SAID GUIDE PRODUCTION

(71) Applicant: Ozgur Sinan YALDIZ, Ankara (TR)

(72) Inventor: Ozgur Sinan YALDIZ, Ankara (TR)

(21) Appl. No.: 18/284,298

(22) PCT Filed: Apr. 11, 2022
(86) PCT No.: PCT/TR2022/050316

§ 371 Date: Sep. 27, 2023

(30) Foreign Application Priority Data
Apr. 26, 2021 (TR) 2021/007090

(51) Int. Cl. A61B 34/10; A61B 17/00; A61B 17/15; A61B 17/17; A61B 17/56; A61B 90/00

(52) U.S. Cl. CPC A61B 34/10; A61B 17/157; A61B 17/1728; A61B 17/1764; A61B 2017/00526; A61B 2017/568; A61B 2034/108; A61B 2090/3762

(57) ABSTRACT

Software based cutting (osteotomy) measuring navigation system processing radiological screen displays used for detecting bone curve for High Tibial Osteotomy (HTO) in Varus and Valgus deformities occurring in knee joint and customised angle adjustable cut and screw guide (7) obtained by means of modelling the values measured by the navigation and transmission thereof to three-dimensioned printer for use in orthopaedics surgery in medicine sector.

### TECHNICAL FIELD

Invention relates to cutting measuring navigation system processing web based radiological screen displays used for detecting bone curve for High Tibal Osteotomy (HTO) in Varus and Valgus deformities occurring in knee joint and customised cut and Screwing obtained by means of modelling the values measured by the navigation and transmission thereof to three-dimensioned printer for use in orthopaedics surgery in medicine sector.

### BACKGROUND OF THE ART

In the state of art said bone curve detection is measured manually on x-ray films and Incision on bone is made by help of wire and markers during operation and manual measurement is made for correction. The nearest technology developed for this purpose is the single use cut guide called PSI Activmotion (https://newcliptechnics.com/contact/) by France based NewClip company. In this technology used by NewClip company, modelling is made from tomography, guide design is customised by normal injection form method. Definition of customised refers to anatomic guide modelling from tomography. In this respect-, it does not have a software based system measuring angular deformities by image processing to plan for each patient separately.

In the state of art new developments to allow processing of x-ray, computerized x-ray, tomography, three-dimensioned tomography images and generate customised guide for each patient are needed.

When the related art is searched. Chinese patent model application numbered CN111481259 A is found. Said application discloses a method of preparing osteotomy guide plate. According to the model magnetic resonance imaging data of bone tissue of the patient are obtained, magnetic resonance imaging data are transmitted to three-dimensioned simulation software and three-dimensioned model of bone tissue is re-generated. However, said system does not disclose a customized process.

As a result, due to above described disadvantages and inadequacy of existing solutions it has been necessary to make development in the related art.

### BRIEF DESCRIPTION OF THE INVENTION

Present invention relates to cutting measuring navigation system processing web based radiological screen displays used for detecting bone curve and customised cut and screw guide obtained by means of modelling the values measured by the navigation meeting the needs mentioned above, eliminating all disadvantages and providing some additional advantages.

Primary purpose of the invention is to develop a technology providing measurement of bone angular deformities of a patient from radiological images, processing the data therefrom and capable to print out customised bone cut and screw guide in three-dimensioned printer. With the invention a separate planning and measurement system is provided for each patient and customised design is provided from the system and thus it will be possible to obtain 3 dimensioned printed bone cut guide and external plate screw guide system modelled by patient's data in order to correct the angular deformity. The invention has the novelty to provide fault free cases without use of conventional surgery guide but with better and customized corrections and cuts.

Another purpose of the invention is to disclose a system to carry standard deformity measurement techniques into digital environment, measure anatomic axis of femur and tibia, define varus or valgus deformity, and automatically calculate angle needed for correction. According to the calculated angle 3-dimensioned cut guide and external implant screw guide is provided.

Under the invention, anatomic and mechanical axis are processed in open source coded software such as open CV, Phyton on radiological image pictures installed into software and after automatic calculation by use of marking and symbols, bone angular deformity of patient is defined as varus or valgus deformity in the software and then it is possible to record the data. In line with the data cut angle to provide correction is discovered and accordingly cut guide design is conducted.

In the invention the data obtained via software are preferably modelled in SolidWorks program and processed according to personal measurement data and then transmitted to 3-dimensioned printer; customized three-dimensioned cut and screw guide to enable physician to screw implant of desired brand in desired angle and size.

In surgery application technique cut guides are convenient for open surgical method. In stage of plating (implant application) mentioned in the invention, skin is completely opened, and screw holes are provided and then plating is made. In this respect the most distinctive feature of the invention is that the distal part of the guide (the part in the lower part) enables fixing implanted plate to patient as external guide with minimally incision on patient's skin.

In order to solve problems existing in the related art and achieve above mentioned purposes, the invention is a method to detect curve in the bone for correction cut in the bone and produce person customized cut and screw guide in the plating method of High Tibial Osteotomy operations in Varus and Valgus deformities occurring in knee joint and comprises process steps of
Transmission of patient's x-ray or tomography film or all radiological images in JPEG format to system having software detecting curve in the bone,
Measurement of mechanic and anatomic axis of femur and tibia in the system having said software and diagnosis of Varus and Valgus deformities and discovery of angle needed for correction,
Modelling of obtained results on person basis and reproduction of cut and screw guide from medically biocompatible material by 3-dimensioned printer.

The structural and characteristic features and all advantages of the invention will be understood better in the figures given below and the detailed description by reference to the figures. Therefore, the assessment should be made based on the figures and taking into account the detailed descriptions.

### FIGURES FOR BETTER UNDERSTANDING OF INVENTION

FIG. 1 shows process of finding femur head centre by help of tangents drawn for femur in FIGS. 1A and 18.

FIG. 2 shows process of finding femur head centre by help of square and diagonals drawn for femur in FIGS. 2A and 28.

FIG. 3 shows process of finding femur distal joint face centre in FIGS. 3A and 3B.

FIG. 4 shows process of finding femur anatomic and mechanical axis in FIGS. 4A and 48.

FIG. 5 shows process of finding tibia proximal joint face mid-point in FIGS. 5A and 58.

FIG. 6 shows process of finding tibia distal joint face mid-point in FIGS. 6A and 6B.

FIG. 7 shows process of finding tibia distal joint face mid-point in FIGS. 7A and 78.

FIG. 8 shows finding tibia axis and their relations in Figures BA, 8B and BC.

FIG. 9 shows view of proximal guide section from various angles.

FIG. 10 shows view of distal guide section from various angles.

FIG. 11 is a general view of guide screw.

FIG. 12 shows general view of cut on tibia bone and screw guide.

FIG. 13 is a detailed view of guide screw.

FIG. 14 shows anatomic and mechanical axis of femur in frontal plan.

FIG. 15 shows drawing of tibia distal joint orientation line in frontal plan in FIG. 15A, drawing of tibia proximal joint orientation line in frontal plan in FIG. 15B.

FIG. 16 shows femur distal joint orientation line in frontal plan.

FIG. 17 shows line combining femur head centre and big trocanter head in FIG. 17A, femur head centre to mid-point of femur neck in FIG. 17B.

FIG. 18 shows relationship between femur proximal joint orientation line in frontal plan to femur mechanical axis in FIG. 18A and femur anatomic axis in FIG. 18B.

FIG. 19 shows relationship between femur proximal joint orientation line in frontal plan and femur anatomic axis.

FIG. 20 shows relationship between femur distal joint orientation line in frontal plan to femur mechanical axis in FIG. 20A and femur anatomic axis in FIG. 208.

FIG. 21 shows relationship between tibia proximal joint orientation line and tibia anatomic and mechanic axis.

FIG. 22 shows relationship between tibia distal joint orientation line in frontal plan and tibia anatomic and mechanic axis.

FIG. 23 shows view of marking and calculation of mechanical axis of lower extremity in frontal plan.

FIG. 24 shows view of marking and calculation of mechanical axis of lower extremity in frontal plan.

FIG. 25 shows view of marking and calculation of mechanical axis of lower extremity in frontal plan.

FIG. 26 shows view of drawing mLDFA angle to detect whether or not deformity occurs in femur in frontal plan.

FIG. 27 shows drawing of occurrence of varus or valgus deformities in femur in frontal plan.

FIG. 28 shows drawing of MPTA angle to detect whether or not deformity occurs in tibia in frontal plan.

FIG. 29 shows varus deformity in tibia if angle in MPTA in FIG. 29A is smaller than 85 degrees and valgus deformity in tibia if MPTA in FIG. 298 is bigger than 90 degrees regarding deformity in tibia in frontal plan.

FIG. 30 shows drawing of JLCA angle to detect whether or not deformity occurs in knee joint in frontal plan.

Regarding deformity in knee joint in frontal plan FIG. 31 shows valgus deformity in knee joint if angle in JLCA in FIG. 31A is bigger than 2 degrees and in medial and varus deformity in knee joint if JLCA in FIG. 318 is bigger than 2 degrees and in lateral.

FIG. 32 shows position of placed plant (implant).

### REFERENCE NUMBERS

1—Guide hole
2—Plate housing
3—Kishner wire guide hole
4—Tibia support flap
5—Locking hole
6—Person customized angle adjustable cut area
7—Cut and screw guide
8—Proximal section
9—Distal section
10—Proximal guide section
11—Distal guide section
12—Guide screw
13—Plate

### DETAILED DESCRIPTION OF THE INVENTION

In this detailed description, the preferred embodiments of the invention have been described in a manner not forming any restrictive effect and only for purpose of better understanding of the matter.

Invention relates to software based cutting measuring navigation system processing radiological screen displays used for detecting bone curve for High Tibial Osteotomy (HTO) in Varus and Valgus deformities occurring in knee joint and customised angle adjustable cut and screw guide (7) obtained by means of modelling the values measured by the navigation and transmission thereof to three-dimensioned printer for use in orthopaedics surgery in medicine sector.

In plating method of High Tibial Osteotomy (HTO-High Tibial Osteotomy) operations for treatment of Varus and Valgus deformities occurring in kneed joint x-ray or tomography film of the patient is transmitted to navigation system having its own software and detecting curve in bone by surgeon in order to detect curve in the bone for recovery osteotomy to be conducted to bone. Then marking directed by navigation system having said software is conducted on x-ray or tomography film added to system. Thus, it is possible to plan osteotomy and deformity in patient's bone in advance. In order to perform osteotomy and recovery work free of fault, a cut and screw guide (7) printed out from bio-compatible material from 3-dimensioned printer by use of obtained data.

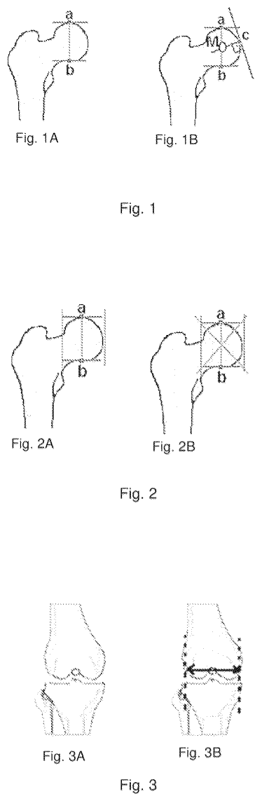

In working principle of said software based system lemur and tibia mechanical and anatomic axis are calculated based on radiological images in the software. It is needed to find out proximal and distal joints centres of femur to draw femur mechanical axis. For these two tangents parallel to each other are drawn from top and bottom for femur (FIG. 1A and FIG. 1B). Points of contacts of tangents with femur head (points a and b) are combined. Thus, diameter of circle is found out.

Then a tangent is drawn from medial, the point where the line from contact point of the tangent with femur head (point c in FIG. 1B) cuts diameter is detected as femur head centre (M). Two tangents are added vertically from medial and lateral to two tangents drawn in FIG. 1 and thus figure is made square (FIG. 2A). Diagonals of square are drawn, and centre is found (FIG. 2B).

Centre of femur distal joint face can be found in two ways.
1. Top point of femoral dent can be taken (FIG. 3A). Femoral dent matches centre of femur distal joint face.
2. Femur condils outer edge distances are measured, and mid-point is taken. This point matches approximately femoral dent top point (FIG. 3B).

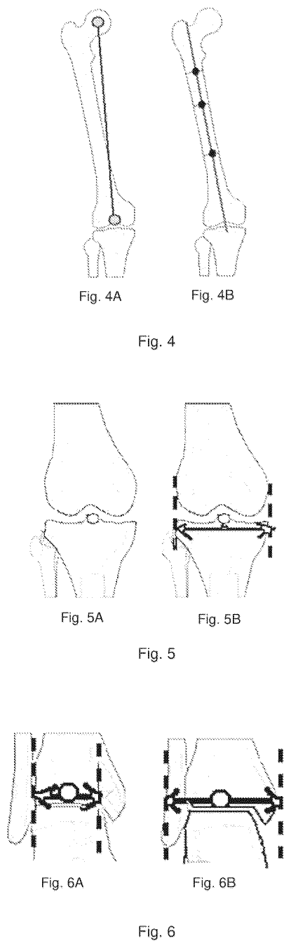

After femur proximal and distal joint face centre points are found for lemur mechanic axis, these two points are connected, and mechanic axis of femur is drawn (FIG. 4A).

Femur anatomic axis for femur anatomic axis is drawn by combining mid points of lines drawn vertically from two or three points to femur diaphysis (FIG. 4B).

Processes mentioned hereunder are processed in software and are calculated by help of software by providing reference signs and symbols on x-ray images.

It is needed to find out proximal and distal joints centres of tibia to draw tibia mechanical axis. Tibia proximal joint face centre is found in two ways.
1. distance between two tibial spines (tubercule) can be taken (Figure SA).
2. midpoint of tibial plate can be taken (Figure SB). For this a line is drawn to joint lace from the point where internal tibial plate finishes. Similarly, a second line is drawn from the point where external tibial plate finishes. Distance between these lines is combined vertically and mid-point centre is shown.

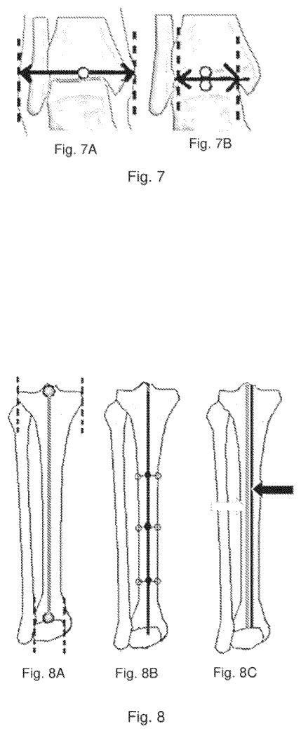

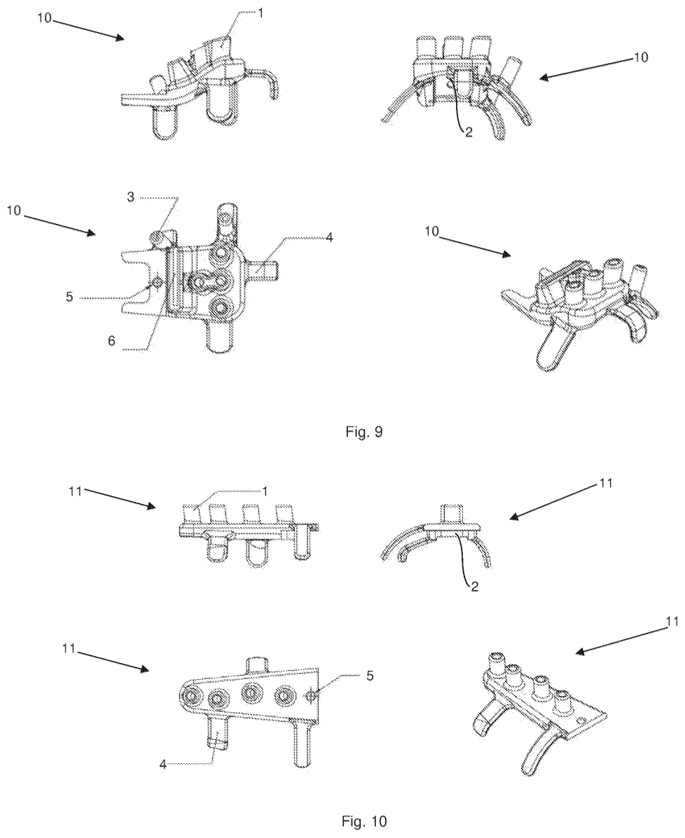

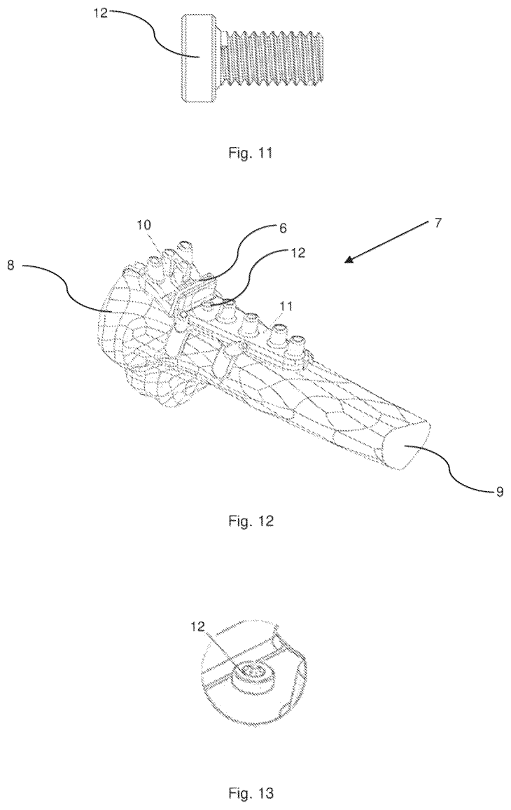

Tibia distal joint face centre is found in four ways.
1. distal tibial joint face mid-point is found (FIG. 6A).
2. Tibia and fibula bones mid-point is found (FIG. 6B),
3. Midpoint of soft tissues is found (FIG. 7A).
4. Mid-point of talus is found (FIG. 7B), Mid-point of talus dorm given in FIG. 7B also corresponds to same point. (Midpoint of talus superior joint face also indicates same point.)

Tibia proximal and distal joint face mid points are combined, and mechanic axis of tibia is drawn (Figure BA).

For Tibia anatomic axis, mid points of lines drawn to diaphysis of tibia from two or three points and these points are combined and tibia anatomic axis is drawn (FIG. 88). FIG. 8 shows the relationship between mechanic axis in FIG. 8A, anatomic axis in FIG. 8B), tibia anatomic (dark arrow) and mechanic axis (light arrow) in FIG. 8C.

Anatomic and Mechanic Axis Relationship in Tibia

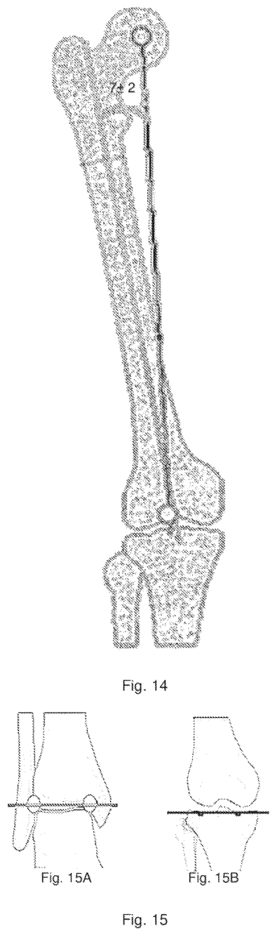

Mechanic axis is a smooth line. Since anatomic axis is the line combining mid points of diaphysis, anatomic axis can be curve (like anatomic axis of femur in sagital plan). Anatomic and mechanic axis of tibia are parallel to each other in frontal plan and there is only a few mm between them. The angle between two axis is 0 degree. For that reason, in practice anatomic and mechanic axis are deemed as the same (FIG. 8C). Anatomic and mechanic axis of femur are different in frontal plan. The angle between two axis is 7 degrees on average. Normally 2 degrees deviation may occur (FIG. 14).

Tibia Joint Orientation Lines

To draw tibia distal joint orientation line in frontal plan, distal tibia subcontral line is taken as basis (FIG. 15A). To draw tibia proximal joint orientation line in frontal plan, concave points of two tibial plate subconoral line are combined in software (FIG. 15B). FIG. 15A shows drawing of tibia distal joint orientation line in frontal plan. FIG. 15B shows drawing of tibia proximal joint orientation line in frontal plan.

Femur Joint Orientation Lines

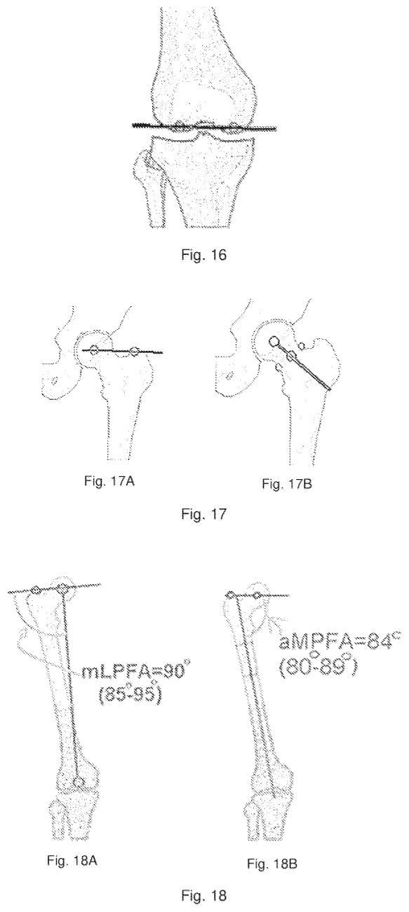

To draw femur distal joint orientation line in frontal plan, distal femur subcondral line is taken as basis and drawn in software (FIG. 16).

Two lines are used for femur proximal joint orientation in frontal plan.
1. Line combining big trocanter top point to femur head centre is drawn in the software (FIG. 17A).
2. Line combining femur neck mid-point to femur head centre is drawn in the software (FIG. 17B).

Relationship Between Joint Orientation Lines and Mechanic and Anatomic Axis

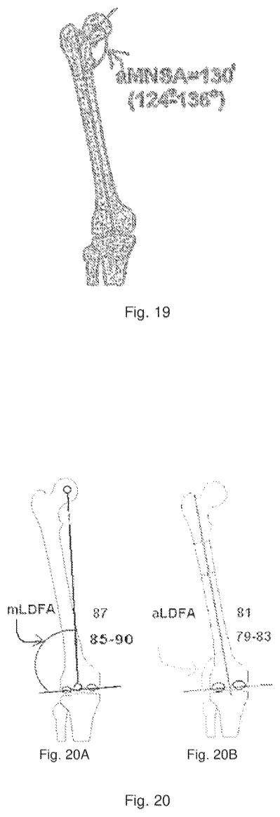

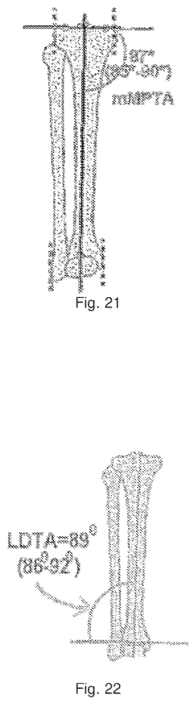

Angles measured to show these relations are defined with 4 capital letters in general. First letter defines direction of angle. If angle is in frontal plan, angle direction is either lateral or medial. If in sagital plan, it is either anterior or posterior. For that reason, first letters is one of L, M, A or P which are initial letters of direction words. Second letter indicates if angle is in proximal or distal of the bone. Second letter is P if in proximal and D if in distal. Third letter indicates where the angle belong to (tibia, femur). If angle is tibia third letter is T and if femur, it is F. Fourth letter is the same in all of them and is initial letter of term angle, which is A: Different from them, a or m in small letter is written before 4-capital letter angle term and a indicates that angle is drawn according to anatomic axis, m to mechanical axis.
1. mLPFA: Line combining femur head centre and trocanter top makes an angle of 90 degrees on average with femur mechanic axis in lateral (minimum 85 and maximum 90 degrees). This angle is called Lateral Proksimal Femoral Açi (mLPFA) (FIG. 18A). It is displayed on monitor in software based system.
2. aMPFA: This line combining femur head centre and trocanter top makes an angle of 84 degrees on average with anatomic axis (minimum 80 and maximum 89 degrees). This angle is called Medial Proksimal Femoral Angle aMPFA (FIG. 18B).
3. aMNSA: This line combining femur head centre and femur neck mid point makes an angle of 130 degrees on average with anatomic axis (minimum 124 and maximum 136 degrees). This angle is called Medial Neck-Shalt Angle aMNSA (FIG. 19).
4. mLDFA and aLDFA: Distal femur joint orientation line makes an angle of 87 degrees on average with femur mechanic axis in lateral (minimum 85 and maximum 90 degrees) (FIG. 20A. This angle is called Lateral Distal Femoral Angle (mLDFA). This line makes an angle of 81 degrees on average with anatomic axis (minimum 79 and maximum 83 degrees) (FIG. 20A). This angle is called anatomic Lateral Distal Femoral Angle (aLDFA). It is drawn and measured in software based system.
5. mMPTA: Proximal tibia joint orientation line makes an angle of 87 degrees on average with tibia mechanic axis in medial (minimum 85 and maximum 90 degrees) (FIG. 21. This angle is called Medial Proksimal Tibial Angle (mMPTA), it is calculated in software based system and displayed on monitor. This line makes same degree angle with anatomic axis in medial. Because anatomic and mechanic effect of tibia is assumed the same.
6. mLDTA: Distal tibia joint orientation line makes an angle of 89 degrees on average with tibia anatomic and mechanic axis in medial (minimum 86 and maximum 92 degrees) in lateral (FIG. 22). This angle is called Lateral Distal Tibial Angle (mLDTA).

Frontal Plan Malalignment Test (MAT)

When a case with deformity doubt is encountered, the first thing to be asked “is there deformity?”. Some deformities are certain without leaving any room for doubt. Most of deformities are recognized only after performance of required measurements. Regardless of the fact that deformity is certain or not, the measurements of deformity (malalignment test) is conducted routinely. Because the obtained data will be needed for processes to be performed later.

Malalignment Test 1

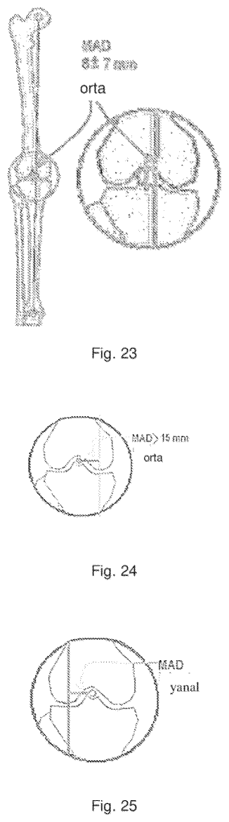

Purpose of this test is to answer of the question “is there deformity?” Centre of femur head and foot ankle is found. These two points are joined and mechanic axis of the lower extremities is drawn. This line passes from 8±7 mm medial on average (FIG. 23).

Passing of lower extremity mechanic axis through knee center from medial up to 15 mm is assumed normal. However, if more than 15 mm of mechanical axis or passing through lateral (regardless of distance), it is called Mechanic Axis Deviation (MAD). If MAD is in medial and bigger than 15 mm, there is varus deformity (FIG. 24). If mechanic axis of lower extremity passes lateral to knee centre (amount is not essential) there is valgus deformity (FIG. 25). Whether or not deformity exists is estimated in software based system and displayed on monitor.

Malalignment Test 2

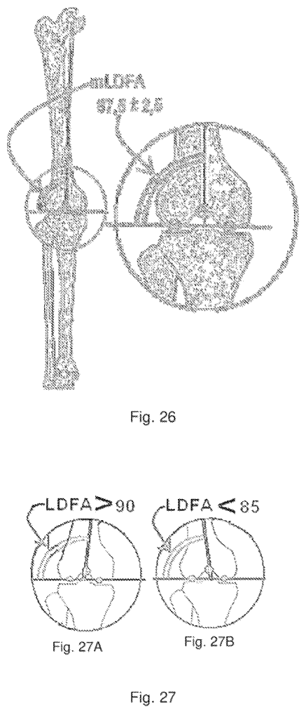

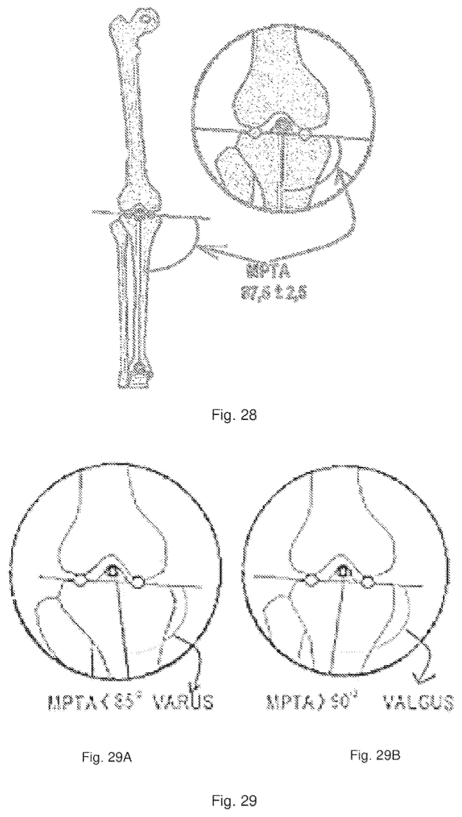

In this test, answer to the question of “Where is deformity; in femur?” is searched. Lateral Distal Femoral Angle (mLDFA) is measured for it. Femur head centre is combined with femur distal joint face centre and femur mechanic axis is drawn. Then femoral condils lowest subcondral points are combined and distal femur orientation line is drawn. These two lines makes an angle on femur outer side (mLDFA). This angle is normally 87.5±2.5 degree (FIG. 26). If this angle is bigger than 90 degrees, then there is deformity in femur and indicates varus deformity. If angle is less than 85 degrees, then there is valgus deformity in femur (FIG. 27). It is marked and computed in software based system.

Malalignment Test 3

In this fest, answer to the question of “Where is deformity; in tibia?” is searched. Medial Proksimal Tibial Angle (MPTA) Is measured for it. Tibia proximal joint face centre is combined with tibia distal joint face centre and tibia mechanic axis is drawn. Then tibial plates lowest subcondral points are combined and proximal tibia orientation line is drawn, these two lines makes an angle on tibia inner side (MPTA). This angle is normally 87.5±2.5 degree (FIG. 28). It is marked and computed in software based system. Il this angle is bigger than 85 degrees, then there is deformity in tibia and indicates varus deformity (FIG. 29A). If this angle is bigger than 90 degrees, then there is deformity in tibia and indicates valgus deformity (FIG. 29B). It is marked and computed in software based system.

Malalignment Test 4

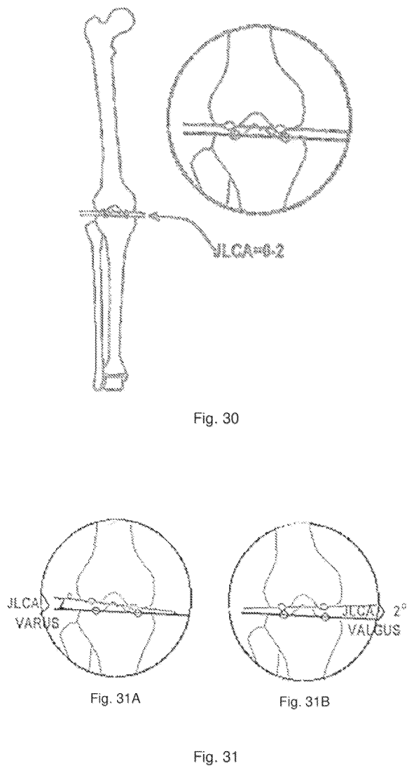

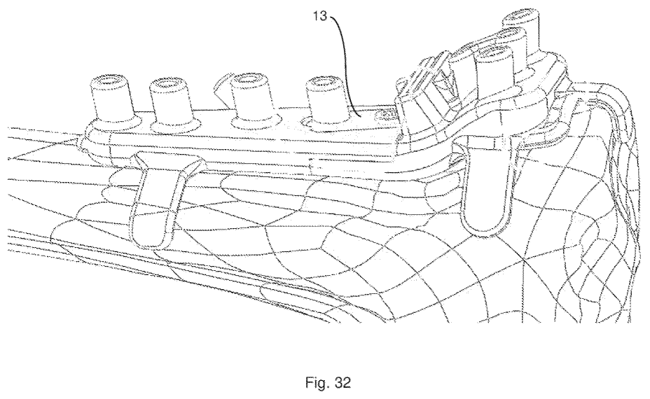

In this test, answer to the question of “Where is deformity; in knee joint?” is searched. JLCA (Joint line convergence angle) is measured between femoral and tibial knee joint lines to answer this question. Femoral condils lowest subcondral points are combined and distal femur orientation line is drawn. Then tibial plates lowest subcondral points are combined and proximal tibia orientation line is drawn, these two lines are parallel to each other. There can be an angle up to 2 degrees between them. Angle bigger than 2 degrees indicates deformity in knee joint (FIG. 30). If this angle is bigger than 2 degrees and in medial, knee joint has valgus deformity (FIG. 31A). If JLCA angle is bigger than 2 degrees and in lateral, knee joint has varus deformity (FIG. 31B). It is marked and computed in software based System.

According to angles measured and computed in software based system and selected plate, customized YTO (High Tibial Osteotomy) and external screw guide is obtained with help of 3-dimensioned printing.

Tibia support flaps (4) are support structures in flap form to provide holding of cut and screw guide (7) onto tibia bone or skin during operation. Similarly, kishner wire guide holes (3) are the holes used to fix cut and screw guide (7) onto said bone, After scraping required muscle and tissue members of tibia bone (FIG. 12), it is fixed onto said bone through tibia support flaps (4) and kishner wire guide holes (3) by help of preferably wire or pin. Also, proximal guide section (10) which is upper part of cut and screw guide (7) is fixed to bone through guide holes (1).

Cut area (6) adjustable tailored to person is the area where saw goes into cut and screw guide (7) for cutting bone (osteotomy). This area is in the angled form according to angle measured in software based system. Saw printed out in person tailored angle conducts osteotomy at the angle specified from bone cut channel. Here physician decides on open and close mixed osteotomy by marking in the software. Thus cuts in angles planned in software based system are conducted.

Proximal guide section (10) and distal guide section (11) are connected to each other through locking hole (5) by means of 3-dimensioned guide screw (12). Said locking hole (5) is the screw hole connecting cut and screw guide (7) proximal guide section (10) and distal guide section (11).

Cut and screw guide (7) proximal guide section (10) is removed from where it is fixe. Plate (13) (implant) designed according to desired brand and model is placed into plate housing (2) in proximal guide section (10) of cut and screw guide (7) and fixed to bone. During fixing implant to bone, tibia support flaps (4) and kishner wire guide holes (3) are used. In this respect, implant housing (2) is the structure where into implant will seat after osteotomy. Thus, the cut and screw guide (7) designed tailored for person at 3-dimensioned printer are used as external screw guide.

Said cut and screw guide (7) is obtained by means of joining proximal guide section (10) and distal guide section (11) and placement of plate (13) (implant) thereinto. In this respect, proximal guide section (10) and distal guide section (11) comprise a plate (implant) housing (2) convenient for placement of implant thereinto.

After implant (13) is placed from the place opened to conduct cut and after conduct of cut and fixation to bone, lower end (distal end) of implant is placed to bone through periost subcutaneously. Guide screw (12) is passed through locking holes (5) located in cut and screw guide (7) and skin is marked from guide holes (1) and locking holes (5) to fix cut and screw guide (7) and small cuts are made by use of bisturi. Firstly, bone is drilled by use of drill to fix implant by screw in the cuts. Guide screw (12) is fixed to implant (13) and bone from guide holes (1) by use of preferably screwdriver or a similar material. Lower tip part of cut and screw guide (7) functions as an external guide to allow implanting by distal guide section (11) minimal invasive method.

Since guide holes (1) located on cut and screw guide (7) are in the same place as implant (13) holes, implant (13) is firstly screwed on upper part by help of proximal guide section (10). It is brought to bone at desired angle by means of angling apparatus based on recovery angle degree measured in software based system and placed in a manner distal guide section (11) remains on in cut and screw guide (7). As stated, distal section of implant is fixed onto bone by help of guide holes (1) and operation is completed with minimally cuts (Minimally Invasive Operation) skin.

As a result, data obtained from said software based system used for bone measurement and cut are preferably processed in web based software, received data are modelled person tailored in preferably solid works program and transferred to 3-dimensioned printer. Cut and screw guide (7) which is designed person tailored at 3-dimensioned printer comprises two basic parts, namely proximal guide section (10) and distal guide section (11). Proximal guide section (10) is fixed to bone and bone is cut in cut and screw guide (7) produced with a person tailored angle. After combining with distal guide section (119, YTO plate (implant) is placed into cut and screw guide (7) and physician adjusts the bone at angle s/he measures, proximal guide section (10) and distal guide section (11), cut and screw guide (7) fix the plate externally (with minimally invasive on to skin) by help of screwing holes on cut and screw guide (7).

1. A method to detect curve in the bone for correction cut in the bone and produce personalized cut and screw guide (7) in the implanting method of High Tibial Osteotomy operations in treatment of Varus and Valgus deformities occurring in knee joint and characterized by comprising process steps of
Transmission of patient's x-ray or tomography film or all radiological images in jpeg format to system having software detecting curve in the bone,
Measurement of mechanic and anatomic axis of femur and tibia in the system having said software and diagnosis of Varus and Valgus deformities and calculate the of angle needed for correction,
Modelling of obtained results on person basis and reproduction of cut and screw guide (7) from medically biocompatible material by 3-dimensioned printer.

2. The method according to claim 1 and characterized by comprising process stop of performance of said modelling by SolidWorks program.

3. A cut and screw guide (7) obtained by a method according to claim 1.

4. The cut and screw guide (7) according to claim 3 and characterized by comprising a proximal guide section (10) and a distal guide section (11).

5. The cut and screw guide (7) according to claim 3 and characterized by comprising tibia support flaps (4) in flap form to provide holding of cut and screw guide (7) onto tibia bone or skin during operation.

6. The cut and screw guide (7) according to claim 3 and characterized by comprising kishner wire guide holes (3) to provide fixation of cut and screw guide (7) to bone.

7. The cut and screw guide (7) according to claim 3 and characterized by comprising cut area (6) with adjustable angle tailored to person where saw goes into cut and screw guide (7) for cutting bone (osteotomy) and angled based on angle measured in software based system.

8. The cut and screw guide (7) according to claim 4 and characterized by comprising plate holes (2) in a manner to place plate (implant) (13) into both proximal guide section (10) and distal guide section (11).
